(19) United States
(12) Patent Application Publication
(10) Pub. No.: US 2011/0253633 A1
(43) Pub. Date: Oct. 20, 2011

(54) CATANIONIC VESICLES, PROCESS FOR PREPARING SAME AND APPLICATIONS THEREOF

(75) Inventors: David Carriere, Montrouge (FR); Youlia Michina, Lyon (FR); Daniel Kopetzki, Berlin (DE)

(73) Assignee: COMMISSARIAT A L'ENERGIE ATOMIQUE ET AUX ENERGIES ALT., Paris (FR)

(21) Appl. No.: 13/125,220

(22) PCT Filed: Oct. 20, 2009
(86) PCT No.: PCT/FR2009/001225

§ 371 Date: Jul. 5, 2011

(30) Foreign Application Priority Data
Oct. 20, 2008 (FR) 0805806

(51) Int. Cl. C02F 1/42; A61K 8/02; A61K 9/48; A61K 8/40; A61K 8/11; A61K 47/18; A61K 9/00

(52) U.S. Cl. 210/679; 514/784; 424/401; 424/400; 424/451

(57) ABSTRACT

Catanionic vesicles, the walls of which are constituted of a mixture of surfactants of opposite charges. Process for preparing same and uses thereof, especially in the field of pharmacy or cosmetics, in the sector for the pollution control of aqueous media or in the field of the synthesis of nanoparticles.

The present invention relates to catanionic vesicles, i.e. vesicles whose walls are constituted of a mixture of surfactants of opposite charge. It also relates to a method of preparation thereof and uses thereof, notably in the field of pharmacy, cosmetics, in decontamination of aqueous environments or in the area of synthesis of nanoparticles.

Mixtures of anionic and cationic surfactants in an aqueous medium give rise to what are generally called “catanionic” solutions.

After ion pairing, counter-ions form a salt in excess and induce increased conductivity of the samples that masks the electrostatic interactions. A particular type of salt-free catanionic formulation is obtained using only H⁺ and OH⁻ counter-ions, in such a way that no excess of salt is formed by mixing the two surfactants (Dubois M. et al., C. R. Acad. Sci. Paris II C, 1998, 1(9) 567-565). The resultant catanionic systems are commonly called “true catanionic systems”.

When catanionic solutions are heated to a temperature above the melting point of the chains, the anionic and cationic surfactants self-assemble in the form of stable micelles of various shapes (spheres, cylinders or even folded bilayers). Depending on the relative proportions of the cationic and anionic constituents, various forms of structures can then be obtained during cooling of these solutions.

Various structures obtained from catanionic solutions have been observed and described, notably in Zemb T. et al., Science, 1999, 283, 816-819; Dubois M. et al., Nature, 2001, 411, 672-675; WO2005/089927; EP-1 846 152.

The various structures described in these documents are salt-free catanionic formulations, obtained using only H⁺ and OH counter-ions. Moreover, these formulations are obtained by a method that comprises a mixing stage, which can be long (1 hour to several days), and a stage of heating of the mixture, which is very short (one minute). These operating conditions lead to structures in the form of membranes, nanodisks, and polyhedra, which display the characteristic of being porous. The exchanges between the interior of the vesicle and the medium in which it is diluted, generally an aqueous solution, are rapid and the substances contained in the internal cavity of the vesicle are quickly diffused to the outside, see Glinel, K. et al., Langmuir, 2004, 20, 8546-8551.

Therefore it is not possible with such vesicles to create a pH gradient between the external medium and the internal cavity of the vesicles, as equilibration of the two media takes place very rapidly. Now, there are organic active principles that can only dissolve in an aqueous medium at acid pH or at basic pH. However, administration of these active principles to a human or to an animal requires placing them in an approximately neutral composition. Therefore there is a need for vesicles permitting encapsulation of these active substances, said vesicles themselves being dissolved in an approximately neutral medium.

Moreover, various structures for delivering active principles, including sustained-release forms, have already been proposed and are based on encapsulation of active ingredients within vesicles. Accordingly, with this aim, a large number of documents of the prior art describe the use of spherical vesicles constituted of one or more lipid bilayers, commonly called liposomes. However, the use of liposomes is not always completely satisfactory, in terms of stability, and because preparation of them employs methods requiring the use of organic solvents, whose use is not always compatible with physiological media. Furthermore, industry is trying to avoid the large-scale use of organic solvents.

In order to solve all of these problems, the applicant has developed novel catanionic vesicles dispersed in an approximately neutral aqueous medium, and whose internal cavity contains a solution that is either acid, or basic. These vesicles notably have the advantage that they are stable, can be prepared easily by a method that forms the object of the present invention and that does not require the use of organic solvents.

The invention relates firstly to non-porous vesicles dispersed in an aqueous composition (external phase), whose walls, which define an internal phase, are constituted of a mixture of at least one anionic surfactant (AS) with counter-ion A⁺ and at least one cationic surfactant (CS) with counter-ion B⁻, (A⁺, B⁻) being different from (H⁺, OH⁻) but with at least A⁺=H⁺ or B⁻═OH⁻. These vesicles offer the advantage that they are slightly permeable and, in the absence of active substances dissolved in their internal cavity, they contain, in their internal cavity, an aqueous solution with pH differing from that of the external phase by at least one unit of pH. The walls of the vesicles of the invention are constituted of anionic and cationic surfactants self-assembled into an approximately spherical, slightly permeable structure.

The term “non-porous or slightly permeable vesicles” means vesicles whose walls are able to prevent the passage of solutes contained in their internal cavity to the external phase or vice versa for sufficiently long periods. The low permeability of the vesicles can be tested experimentally by measuring, at different time intervals, the concentration of protons (via the pH) or of Cl⁻, Br⁻ ions in the vesicles in contact with a dialysis bath, or by measuring the concentration of a solute encapsulated in the vesicles added during preparation (for example the molecules mentioned below). The variation of the concentration cₛₒₗᵤₜₑ(t) of the solute tested (H⁺, Br⁻, Cl⁻, other compound) is generally given by an exponential law of the type:

$${c}_{solute} ( t ) = {c}_{0 , solute} {e}^{- \frac{3 {P}_{solute}}{R} t}$$

where R is the average radius of the vesicles, t is the time elapsed between encapsulation of the solute and measurement, Pₛₒₗᵤₜₑ is the permeability of the vesicles to the solute in question. The low permeability of the vesicles of the invention for a solute is confirmed by a permeability measurement of less than 10⁻¹⁰ cm/s. For example, for vesicles with a typical diameter of 5 μm, this corresponds to loss of half of the solute in a time greater than 160 hours.

In general, the permeability of a given vesicle for a given solute can differ depending on the direction of passage of the solute. Notably, for a charged solute passing through the wall of a vesicle having a transmembrane potential Δφ=φₑₓₜₑᵣₙₐₗ−φᵢₙₜₑᵣₙₐₗ, the permeability Pₒᵤₜ characterizing exit of the solute and the permeability Pᵢₙ characterizing entry of the solute are connected by:

$$\frac{{P}_{out}}{{P}_{in}} = exp ( - \frac{q Δ φ}{kT} )$$

where q is the charge of the solute, k is Boltzmann's constant and T is the temperature in kelvins. Thus, several orders of magnitude of difference can be observed between the permeability of a charged solute in one direction and in the other direction. A vesicle will be regarded as having low permeability to a solute if at least one of the two permeabilities, in one direction and in the other direction, is less than 5 10⁻¹¹ cm/s. The vesicles of the invention have a permeability to H⁺ or to OH⁻ which is less than 5 10⁻¹¹ cm/s.

The vesicles of the invention are also characterized by:

the fact that a pH difference of at least 1 unit and advantageously 2 units between the internal phase and the external phase of the vesicles is maintained for a time of at least 1 month, and preferably 2 months;

their resistance to dialysis: the vesicles are resistant to dialysis, i.e. they are not destroyed by dialysis (observations by microscopy), their composition of surfactants does not change (observed by HPLC), even if they are left to dialyse for 2100 hours. This is a very surprising phenomenon, because a person skilled in the art would expect dialysis to remove the soluble surfactant, and therefore lead to complete destruction of the vesicles.

Moreover, it was found that, most often, the bilayer of the vesicles is crystalline. This can be observed by X-ray diffraction. This implies that the surfactants are “gelled” in the bilayer, do not exchange with the external solution, and therefore the vesicles are solid.

The invention also relates to a method for preparing a dispersion of vesicles in an aqueous medium, said method comprising the following stages:

i) mixing, in water, at least one anionic surfactant with a counter-ion A⁺ and at least one cationic surfactant with a counter-ion B⁻, (A⁺, B⁻) being different from (H⁺, OH⁻) but with at least A⁺=H⁺ or B⁻═OH⁻;

ii) heating this mixture, for a time tₕₑₐₜ at a temperature T below the melting point of the catanionic mixture and which is greater than or equal to 40° C. but below 90° C., T and tₕₑₐₜ being sufficient to obtain dissolution of the surfactants;

iii) dialysis of the suspension obtained in ii) so as to remove the salts from the external phase.

In more detail:

The method of the invention is carried out in an aqueous medium, i.e. in water or in a mixture of water and another solvent miscible with water. For example, it is possible to use mixtures of water with glycerol (up to 30 vol. % relative to the volume of water) or with DMSO (up to 3 vol. % relative to the volume of water). Following preparation, the vesicles also withstand addition of ethanol, of DMSO or of glycerol in moderate amounts (less than 1 vol. % relative to the volume of water).

Preferably, the method of the invention is carried out in water.

The surfactants are selected as follows:

According to a first variant, which relates to vesicles with an acid internal phase, the anionic surfactant has the H⁺ counter-ion and the cationic surfactant has the B⁻ counter-ion, B⁻ being different from OH⁻.

According to a second variant, which relates to vesicles with a basic internal phase, the anionic surfactant has the A⁺ counter-ion, A⁺ being different from H⁺ and the cationic surfactant has the OH⁻ counter-ion.

The anionic surfactants that can be used according to the invention for formation of vesicles have an anionic polar group and at least one C₈-C₃₀ alkyl chain optionally substituted with one or more hydroxyl groups and one or more halogen atoms. The anionic polar group is selected from acid or acid salt functions. Preferably these anionic surfactants are derivatives with a C₈-C₂₄ alkyl, or C₈-C₂₄ hydroxyalkyl, or C₈-C₂₄ fluoroalkyl chain. Preferably they are selected from carboxylic acids with C₈-C₂₄ alkyl or hydroxyalkyl chain, or C₈-C₂₄ fluoroalkyl, dicarboxylic acids with a hydrophobic C₈-C₂₄ carbon chain, phosphates, sulphates and sulphonates bearing one or two C₈-C₂₄ alkyl chains.

The anionic surfactant used in the invention can be a neutral acid that releases a proton in water. Thus, the expression “anionic surfactant” includes surfactants ionizable to anionic surfactant.

Among these anionic surfactants, in particular we may mention fatty acids such as lauric acid, myristic acid, palmitic acid, dodecanedioic acid, tetradecanedioic acid, hexadecanedioic acid, 12-dodecanoic acid, 14-tetradecanoic acid, 16-hydroxyhexadecanoic acid.

The anionic surfactant can optionally be mixed with a nonionic surfactant, preferably selected from derivatives with C₈-C₂₄ alkyl chain, or C₈-C₂₄ hydroxyalkyl, or C₈-C₂₄ fluoroalkyl, for example a fatty alcohol or a fatty alcohol derivative: a C₈-C₂₄ hydroxyalkyl, an ether of a C₄-C₂₄ carbohydrate and of a C₈-C₂₄ hydroxyalkyl. The nonionic surfactant can be introduced without damaging the vesicles. The use of a nonionic surfactant mixed with the ionic surfactant makes it possible to decrease the pH gradient between the internal compartment and the external medium.

The anionic surfactant either has the H⁺ counter-ion (first variant), or a counter-ion different from H⁺, and in this case the counter-ion can be selected from the salts of alkali metals and alkaline-earth metals, for example Na⁺, K⁺, Li⁺, as well as the organic cations such as NH₄⁺, NH₃⁺OH, NH₃⁺CH₂OH (second variant). During application of the first variant, when we wish to alter the internal pH of the vesicles, we select the use of a surfactant with a counter-ion H⁺ and at least one other counter-ion. For example, it is possible to use a mixture of counter-ions, such as H⁺ and Na⁺.

The cationic surfactants that can be used for forming the vesicles of the invention are molecules having a cationic polar group and at least one alkyl or aryl or aralkyl chain, saturated or unsaturated, having from 8 to 30 carbon atoms and optionally interrupted by an ester or ether function. These surfactants are preferably selected from the monocatenary and bicatenary ammoniums corresponding to the following formulae (I) and (I′) and the corresponding bases:

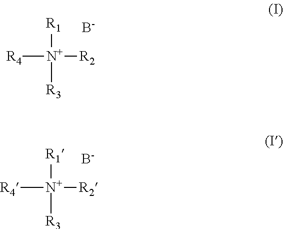

in which:
R₁, R₂ and R₃, R′₁ and R′₂, which may be identical or different, represent a group selected from: H, a C₁-C₄ alkyl, a C₁-C₄ hydroxyalkyl and an alkyl(C₁-C₄)ether,
R₄, R′₃ and R′₄, which may be identical or different, represent a group selected from: a saturated or unsaturated C₈-C₂₄ hydrocarbon chain, a benzyl, a C₈-C₂₄ aralkyl or an alkyl(C₄-C₂₀) ester of alkyl(C₄-C₂₀) group,
B⁻ represents an ion selected from: OH⁻ (second variant), F⁻, Cl⁻, Br⁻, I⁻, BF₄⁻, SO₄²⁻ (first variant).

The cationic surfactant used in the invention can be a neutral base that undergoes protonation in water. Thus, the expression “cationic surfactant” includes the surfactants ionizable to cationic surfactant.

During application of the second variant, to alter the internal pH of the vesicles, it is possible to use a cationic surfactant with a mixture of counter-ions, for example OH⁻ and Br⁻.

In the above formulae (I) and (I′), R₁/R₂, R₃, R′, and R′₂ preferably represent the methyl radical.

In the above formulae (I) and (I′), R′₃, R′₄ and R₄ are notably selected from:
C₈-C₂₄ alkyl chains, for example the stearyl, cetyl, dodecyl and tetradecyl or myristyl chains;
alkyl(C₄-C₂₀) ester of alkyl(C₄-C₂₀) groups, for example alkyl(C₁₆) esters of alkyl(C₄-C₂₀) and alkyl(C₁₂)esters of alkyl(C₄-C₂₀).
B is advantageously selected from: OH⁻ (second variant), and Cl⁻, Br⁻ (first variant).

It is possible, according to the invention, to combine any type of anionic surfactant with any type of cationic surfactant provided the counter-ions are different from the pair (H⁺, OH⁻).

The amounts of each category of surfactants in the starting aqueous medium are given as the number of moles n_{A} and n_{C}, denoting respectively the number of moles of anionic surfactant and the number of moles of cationic surfactant.

In the case of the first variant, the mole fraction of anionic surfactant is calculated from the formula:

$${r}_{A} = \frac{{n}_{A}}{{n}_{A} + {n}_{C}}$$

The fraction r_{A} is optionally selected for each pair of surfactants, or mixtures of surfactants, according to the following protocol:

a) mix the anionic and cationic surfactants together in water, selecting a fraction r_{A1} at random, preferably with an excess of cationic surfactant;

b) heat the mixture to a temperature near, but below the melting point of the anionic surfactant for a time greater than or equal to one hour;

c) submit the suspension obtained to dialysis;

d) during dialysis, measure the variation of r_{A1} in the composition, for example by monitoring the concentration of the surfactants by HPLC;

e) observe that r_{A1} reaches a limit value that no longer changes and is independent of the value of r_{A1} chosen initially;

f) call this value r_{1(A)};

g) prepare the vesicles using a value of r_{A} greater than r_{1(A)}.

In the case of the second variant, the mole fraction of cationic surfactant is calculated from the formula:

$${r}_{C} = \frac{{n}_{C}}{{n}_{A} + {n}_{C}}$$

The fraction r_{C} is selected for each pair of surfactants, or mixtures of surfactants according to the following protocol:

a) mix the anionic and cationic surfactants together in water, selecting a fraction r_{C1} at random, preferably with an excess of anionic surfactant;

b) heat the mixture to a temperature near but below the melting point of the cationic surfactant T_{C}, for a time greater than or equal to one hour;

c) submit the suspension obtained to dialysis;

d) during dialysis, measure the variation of r_{C1} in the composition, for example by monitoring the concentration of the surfactants by HPLC;

e) observe that r_{C1} reaches a limit value that no longer changes and is independent of the value of r_{C1} chosen initially;

f) call this value r_{1(C)};

g) prepare the vesicles using a value of r_{C} greater than r_{1(C)}.

The concentration of surfactants in the mixture is designated c and is calculated as follows:

$$c = \frac{mA + mC}{mA + mC + {m H}_{2} O}$$

m_{A} denoting the mass of anionic surfactant, m_{C} the mass of cationic surfactant and M_{H2O} the mass of water.

The choice of concentration by mass c makes it possible to control the final size of the vesicles: as c decreases, the size of the vesicles increases. Preferably c is between 0.01 and 3%, and even more preferably between 0.33 and 1%. The size of the vesicles is an important parameter which it is useful to be able to control, notably with a view to application in the pharmaceutical sector. Thus, vesicles of small size are more easily assimilated by the human or animal organism.

The suspension prepared in stage i) is heated at a temperature T for a time tₕₑₐₜ.

T is below the melting point of the catanionic mixture, i.e. the melting point of the mixture of the two surfactants, anionic and cationic. It is near but below the melting point of the less soluble of the anionic surfactant and cationic surfactant. T is selected as follows: it must be high enough to permit dissolution of the crystals of insoluble surfactants (the anionic and optionally nonionic surfactants in the case of the first variant). If T is too low, dissolution does not occur, or is too slow, leading to the formation of crystals, of a precipitate or of a supernatant. If T is too high, the solution becomes almost transparent, which means that there is formation of mixed micelles or of very small vesicles which will be removed by dialysis. In practice T is between the melting points of each of the surfactants of the suspension, and generally is greater than or equal to 40° C. T is therefore below the melting point of the least soluble surfactant. Usually the suspension is heated to a temperature T that is less than or equal to 80° C. In practice T is 2 to 5° C. lower than the melting point of the least soluble surfactant.

The heating time tₕₑₐₜ must be long enough to ensure dissolution of the crystals of insoluble surfactants (anionic and optionally nonionic). If tₕₑₐₜ is too long, formation of crystals, of a precipitate or of a supernatant is observed. Visual inspection permits observation of the time required for dissolution of the surfactants. The time required for complete dissolution depends on the surfactants selected. For example a time from one hour to one week can be selected, and a time of one to two days commonly proves satisfactory for many of the mixtures of surfactants.

With a properly selected temperature T, after the time tₕₑₐₜ a white solution is generally obtained, without aggregates or crystals that are visible to the naked eye. The choice of T and of the time tₕₑₐₜ can be refined by checking by microscopy, after dialysis, for the formation of vesicles and absence of residual crystals.

After this stage, the dispersion can optionally be diluted by adding a solvent, preferably water, at the temperature T. This stage is optional, and makes it possible for example to control the final viscosity of the solution of vesicles.

If we wish to encapsulate an active substance in the vesicles, a solution containing the active substance to be encapsulated can be added during stage ii). This solution is then introduced at temperature T.

At the end of this stage, the dispersion is cooled to room temperature (about 18-25° C.). The vesicles that formed during stage ii) are of approximately ovoid, elongated and flat shape.

The dispersion is submitted to dialysis for a time t_{dial}. According to a first variant the dialysis bath is of pure water. Dialysis makes it possible to remove, from the external phase of the dispersion, the salts A⁺B⁻, and in particular the salts H⁺B⁻ (first variant) or A⁺OH⁻ (second variant). These salts formed in the aqueous medium outside the vesicles and in the internal cavity of the vesicles. The method of the invention makes it possible to obtain, at the end of stage ii), non-porous, slightly permeable vesicles of approximately spherical shape and the dialysis stage makes it possible to remove the salts from the aqueous solution in which the vesicles are suspended, or are dispersed. However, the salts that formed inside the cavity of the vesicles are not removed by dialysis, which creates, at the end of dialysis, a pH difference between the aqueous phase in which the vesicles are dispersed and the internal cavity of the vesicles. In the absence of active substance to be encapsulated, this pH difference is greater than one unit of pH, and preferably greater than two units of pH, advantageously greater than three units.

According to a second embodiment of the invention, the pH gradient between the interior of the vesicles and the external phase can be adjusted by using a slightly acid or slightly basic aqueous solution as the dialysis bath, for example an aqueous HCl solution at 0.1 mM or an aqueous NaOH solution at 0.1 mM. Preferably the solution used as dialysis bath according to this embodiment is an aqueous solution of an acid, such as HCl, with concentration in the range from 0 to 0.1 mM or an aqueous solution of a base, such as NaOH, with concentration in the range from 0 to 0.1 mM.

The time t_{dial} is generally selected so that there is complete removal of the ions outside of the vesicles. Usually a time of 2 to 3 days is sufficient. If t_{dial} is too short, the dispersion is unstable and can form a precipitate or a supernatant of aggregates, or can form crystals. If the choice of t_{dial} is too long, this does not alter the quality of the vesicles obtained. However, after several months of dialysis, the vesicles end up losing their contents.

The solution is then withdrawn from the dialysis cassette and stored. The final composition is extremely stable, the vesicles remain intact for several years of storage after removal from the dialysis cassette. They can even remain under dialysis without being destroyed. The solution can be diluted. Tests with dilution by a factor of 50 were performed without notable destruction of the vesicles.

If this method is applied to the pairs of catanionic surfactants that were described in the prior art and notably in WO2005/089927 and EP-1 846 152, formation of porous vesicles is observed at the end of stage ii). As these catanionic surfactants have counter-ions (H⁺, OH⁻), they lead, furthermore, to the formation of vesicles whose internal cavity has a neutral pH, like the external phase.

Document WO2006/070095 indicates that it is possible to use cations with a counter-ion different from OH⁻. However, the vesicles obtained by this method are different from those of the invention.

In fact, the method of the invention differs from that of the prior art by dialysis, the heating temperature and the heating time. In the conditions of the prior art, even if vesicles formed, they would be porous, and in particular, in the absence of dialysis, the preparation is unstable, i.e. there is formation of crystals, a precipitate or a supernatant.

According to the first variant, if active substance to be encapsulated has not been added, it is found that the interior of the vesicles is at a pH below the pH outside the vesicles (typically, pH ˜3, or 1 mM of HBr or HCl, for example). And according to the second variant, if active substance to be encapsulated has not been added, it is found that the interior of the vesicles is at a pH above the pH outside the vesicles.

The internal pH can be measured by fluorescence spectroscopy, time-resolved or static after adding a suitable fluorescent probe.

A pH gradient is maintained for periods of the order of several months. This pH can be adjusted to some extent by varying the size of the vesicles, as was described above, smaller vesicles having internal cavities with lower pH. The pH can also be varied by using a mixture of fatty acid and salt of fatty acid as described above for the first variant. The use of a salt of fatty acid tends to replace HCl or HBr with NaCl or NaBr in the same proportions, and to increase the internal pH. In the case of the second variant, it is possible to use a mixture of ammonium halide and ammonium hydroxide, or of ammonium halide and protonatable ammonium.

The catanionic vesicles of the invention have a size of 0.1-100 μm, adjustable by varying the concentration by mass c of the starting composition of surfactants.

At the end of the method of the invention, the pH inside the vesicles is different from the pH outside the vesicles. This characteristic is obtained directly, without the addition of buffer solution for example. Usually, formation of vesicles with a pH gradient requires addition of a buffer solution which diffuses into the vesicle (liposome), and the excess is then cleaned (C. Grabielle-Madelmont, et al., Journal of Biochemical and Biophysical Methods, 56(1-3): 189-217, June 2003, or K. Holmberg, D. Shah, and M. Schwuger, editors. Handbook of Applied Surface and Colloid Chemistry, volume 2, Wiley-VCH, New York, 2002). In the method of the invention, the solution is acidified or alkalized naturally when the vesicles form. The retention times of the acid or basic solution in the internal cavity of the vesicles are comparable, or even greater, than the preparations of the prior art.

According to one embodiment, the vesicles of the invention can be stabilized by at least one polymer adsorbed on their surface. Among the polymers that can be used, we may mention those selected from:
polysaccharides, for example dextrans and derivatives of cellulose such as hydroxymethylcelluloses, hydroxyethylcelluloses and hydroxypropylcelluloses,
synthetic polymers such as polyethylene glycols (PEG), polyvinylpyrrolidone (PVP), polystyrenes, polyacrylates, polyvinyl alcohols such as the products sold under the trade names PVA, Ethenol®, Poval®, Acroflex®, Airvol®, Alcotex® or Aquafilm®.

Among these polymers, it is preferred quite particularly to use slightly adsorbent polymers such as polyoxyethylene, dextran, polyvinylpyrrolidone (PVP), ethoxylated diblock polymers such as the polymers sold under the trade name Varonic® by the company Degussa-Goldschmidt, block copolymers based on ethylene oxide and propylene oxide such as the polymers sold by the company BASF under the trade names Pluronic® and Lutrol® and their water-soluble equivalents, water-soluble triblock copolymers, i.e. copolymers composed of hydrophilic-hydrophobic-hydrophilic blocks such as the products sold under the names Methyl-Oxyrane, EOPO copolymer, Pluronic®, Antarox®, Arcol®, Daltocel®, Dowfax® and their analogs with polystyrene as the hydrophobic group.

When they are used, these polymers preferably represent from 50 to 400 wt. % and even more particularly from 100 to 200 wt. % relative to the total weight of the bilayer.

Coating of the vesicles of the invention is not necessary but may be useful when we wish to introduce them into a medium strongly loaded with salts, in order to ensure their stability.

It is possible to encapsulate water-soluble active principles in the vesicles with excellent yields: up to 66% for active principles of charge opposite to that of the internal cavity of the vesicles, 30% for active principles of charge identical to that of the internal cavity of the vesicles, 10% for neutral active principles. In the liposomes of the prior art, far lower yields are usually quoted. In particular, in other catanionic systems, without a pH gradient between the internal phase and the external phase, yields of up to 70% for solutes of charge opposite to the vesicles, and less than 8% for solutes of the same charge as the vesicles, are reported in the literature.

The retention times of the vesicles of the invention are comparable or greater than the times usually reported for the vesicles of the prior art.

Vesicles without an encapsulated solute but which have an acid or basic interior at the end of the method of the invention are capable of spontaneously absorbing certain cationic or anionic compounds, respectively, which are then encapsulated inside the vesicles. Moreover, the walls of the vesicles are positively charged in the case of the first variant, and are therefore able to attract compounds of opposite charge, which then become concentrated near the walls of the vesicles. According to one variant, the method of the invention comprises a stage during which an active substance to be encapsulated is added to the composition following stage iii).

These properties mean that the vesicles of the invention can be used for encapsulating active principles of the water-soluble organic molecule type of low pH (first variant) or of high pH (second variant), notably in the medical or pharmaceutical field and in cosmetics. These vesicles are stable over periods of several months and notably make it possible to retain, by adsorption and/or encapsulation, and to control the slow diffusion of active pharmaceutical or cosmetic molecules or even cells, such as bacteria for example.

These properties also make it possible to envisage the use of the vesicles of the invention for trapping ions in an aqueous medium, and this use finds applications notably in the decontamination of aqueous environments. The method of the invention makes it possible to produce vesicles that attract positively and negatively charged species simultaneously, which is particularly interesting for applications in water decontamination.

These properties also allow us to envisage the use of the vesicles of the invention for trapping metal ions in the internal compartment of the vesicle, which can react with the B⁻ ions to form particles of small size, for example nanometric. For example, with B⁻=Cl⁻ or Br⁻ it is possible to react Ag⁺ ions, or with B⁻═OH⁻ (second variant) it is possible to react transition metals (Cu²⁺, Mn²⁺, Zn²⁺ etc.). Any catalysed hydrolysis-condensation reaction in an acid or basic aqueous medium, for example hydrolysis-condensation of tetraethoxysilane to SiO₂, can be envisaged.

Finally, the invention also relates to the use of at least one vesicle as described above, for formulation of active species in a cosmetic composition or in a pharmaceutical composition.

A cosmetic composition comprising at least one vesicle as described above constitutes another object of the invention.

A pharmaceutical composition comprising at least one vesicle as described above constitutes another object of the invention.

In more detail, and for illustration of the invention, examples are given below of surfactants that have led effectively to the formation of vesicles according to the invention by means of the method described above.

Implementation of the first variant:
The solvents tested were: H₂O, D₂O, a water-glycerol mixture (70:30)
Anionic surfactants:

They can be selected from the following fatty acids and salts of fatty acids:

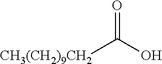

Lauric acid 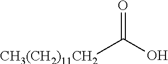
Myristic acid 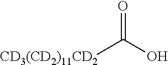
Deuterated myristic acid 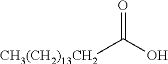
Palmitic acid 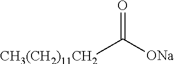
Sodium myristate

Complex fatty acids:

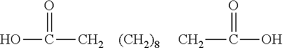

Dodecanedioic acid 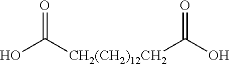
Hexadecanedioic acid 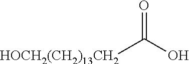
16-Hydroxydecanoic acid

Cationic surfactants:

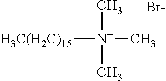

Cetyl trimethylammonium bromide 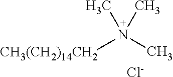
Cetyl trimethylammonium chloride 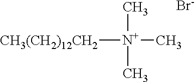
Myristyl trimethylammonium bromide

Other surfactants:

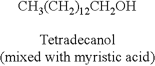

Cationic solutes:

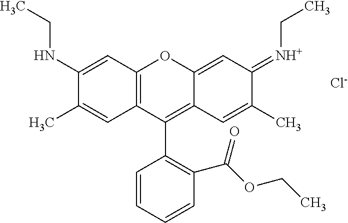

Chloride of Rhodamine 6G 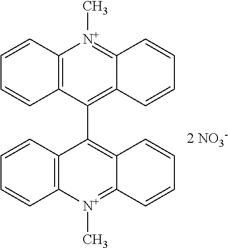
Lucigenin

Neutral or amphoteric solutes:

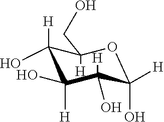

Glucose 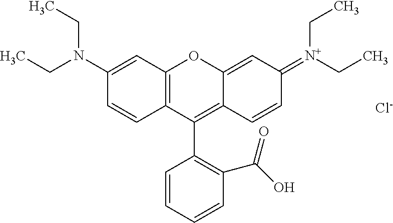
Chloride of Rhodamine B

Anionic solutes:

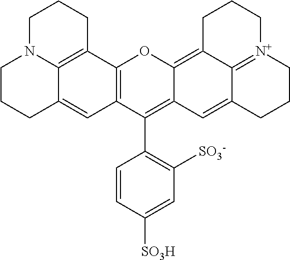

Sulforhodamine 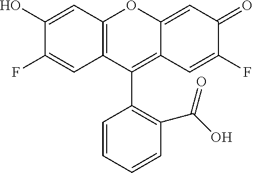
Oregon Green

Stage i): prepare a suspension of water, of cationic surfactant and of anionic surfactant, optionally of solute to be encapsulated, taking suitable values of r and c, with

$$r = \frac{n ( anionic )}{n ( anionic ) + n ( cationic )}$$

(mole fraction of anionic surfactant)

$$c = \frac{m ( anionic ) + m ( cationic )}{m ( anionic ) + m ( cationic ) + m ( water )}$$

(concentration by mass of surfactant)

The choice of r is such that vesicles are effectively obtained at the end of dialysis. The method for efficiently determining the possible values of r was described above. r can be determined by various tests, but we chose the following method: preparation is carried out as explained above, choosing r at random (preferably so that there is a significant excess of water-soluble surfactant, in this case cationic). The variation of r is measured during dialysis, for example by separately measuring the concentrations of the two surfactants by HPLC. It is found that r eventually reaches a limit value that no longer changes, and which is independent of the r chosen initially. The vesicles are then prepared at this molar ratio, or with compositions richer in insoluble surfactant (here anionic or nonionic).

For example, in the case of myristic acid/cetyltrimethylammonium chloride or bromide (CTACl or CTABr) mixture, the composition of the samples prepared at any values of r stabilizes at r˜0.60 in several hundred hours of dialysis. We then decide to prepare the vesicles at a molar ratio richer in myristic acid, i.e. r=0.66. It is noted that preparations carried out at higher r remain intact and also form vesicles. The same type of behaviour is obtained with other mixtures of surfactants.

The choice of c makes it possible, to some extent, to control the final size of the vesicles: as c decreases, the size of the vesicles increases. There are no particular limits on the amount of solute to be added, except that it has been found that very high concentrations of certain solutes can prevent the vesicles forming.

A value c=1% is generally chosen.

If we decide to encapsulate a solute, it is added at this stage so that the final concentration is preferably in the range 30 μM-50 mM. Lower, and higher, concentrations can be envisaged. However, in certain cases when the concentration is too high, it is found that addition of solute prevents formation of vesicles.

An aqueous suspension is then obtained, which usually has crystals of insoluble surfactant (for example myristic acid), and optionally the solute to be encapsulated.

Stage ii): this suspension is heated at temperature T for a time tₕₑₐₜ.

T is selected as follows: it must be high enough to permit dissolution of the crystals of insoluble surfactants. If T is too low, dissolution does not occur, or is too slow, leading to the formation of crystals, of a precipitate or of a supernatant. If T is too high, the solution becomes almost transparent, which means there will be formation of mixed micelles or of very small vesicles which will be removed by dialysis.

tₕₑₐₜ must be long enough to ensure dissolution of the crystals of insoluble surfactant.

If T is chosen well, after the time tₕₑₐₜ a white solution is generally obtained without aggregates or crystals visible to the naked eye. The choice of T and tₕₑₐₜ can be refined by checking by microscopy that the vesicles formed properly after dialysis and that there are no residual crystals.

For example, in the case of the myristic acid/CTABr or CTACl mixture, heating can be carried out at 50° C. for 2 days regardless of the choice of r.

The suspension is optionally diluted preferably by adding water at temperature T, or a solution containing the solute to be encapsulated. This stage is optional, and for example makes it possible to control the final viscosity of the solution of vesicles. For example, with the myristic acid/CTACl or myristic acid/CTABr mixture, with r=0.66 and c=1%, after dialysis we obtain a very viscous solution (gel) of vesicles with diameter of around 5 μm. If the solution is diluted by a factor of 3 before dialysis, a fluid solution is obtained, which contains vesicles with a diameter of around 5 μm.

After cooling, the suspension is submitted to dialysis for a time t_{dial}.

For example, dialysis cassettes with a molecular cut-off threshold of 10 kDa are used, but lower thresholds are also suitable. The dialysis bath can be changed for fresh water at regular intervals, notably to remove the solute that has not been encapsulated in the vesicles, but this is optional. It is preferable to agitate the dialysis bath to permit rapid removal of the salt between the vesicles, and thus avoid their destabilization. t_{dial} is generally selected such that removal of the ions between the vesicles (not inside) is complete. Usually a time of 2 to 3 days is sufficient.

During dialysis, often phase separation is observed first (typically after 12 hours). It is then sufficient to agitate the cassette a little so that the solution homogenizes, and finally a suspension is obtained which never separates into two phases again.

The solution is withdrawn from the dialysis cassette and stored. The final solution is extremely stable, the vesicles remain intact after several years of storage after removal from the cassette, and they can even remain under dialysis without being destroyed. The solution can be diluted by a factor of 50 without notable destruction of the vesicles.

If solute to be encapsulated has not been added, it is found that the interior of the vesicles is at a lower pH than outside the vesicles (typically, pH ˜3, i.e. 1 mM of HBr or HCl).

A pH gradient is maintained for times of the order of several months (half-life of HCl=100 days). This pH can be modulated by varying the size of the vesicles, as described above, smaller vesicles producing lower pH. The pH can also be varied by using a fatty acid/salt of fatty acid mixture as described above. The use of salt of fatty acid tends to replace HCl or HBr with NaCl or NaBr in the same proportions. The internal pH can be measured by fluorescence spectroscopy, time-resolved or static after adding a suitable fluorescent probe.

Optionally, solute to be encapsulated can be added. If the vesicles were prepared without addition of solute in the preceding stages, they are then able to absorb certain compounds present in solution, for example Rhodamine B or Rhodamine 6G. It is sufficient to add the solute and wait about ten minutes before the vesicles absorb the solute spontaneously.

If a solute was added during preparation of the vesicles, it is found that a substantial fraction remains within the vesicles: up to 66% if it is anionic, of the order of 30% if it is cationic or neutral. The vesicles lose the solute very slowly, with half-lives of the order of a hundred days, with variations depending on the nature of the solute. Presence of the solute can be verified by the usual spectroscopic techniques. It is optionally possible to employ more elaborate techniques, which show that the solute present in the final solution actually is inside the vesicles, not outside (confocal microscopy, time-resolved fluorescence spectroscopy). The cationic solutes are uniformly distributed inside the vesicles, and the anionic solutes are concentrated near the walls of the vesicles, only on the inside of the wall.

Implementation of the second variant:

The following surfactants were tested:
Anionic surfactants:

Sodium myristate, sodium tetradecane sulfonate, sodium hexadecane sulfonate.
Cationic surfactants: they are selected from the following amines and salts of amines:

Tridecylamine, tetradecylamine, hexadecylamine, N,N-dimethyltridecylamine, N,N-dimethyltetradecylamine.

### EXPERIMENTAL SECTION

### Drawings

FIG. 1: myristic acid/CTACl vesicles r=0.65 c=1.0% dialysed for 5 days, observation with confocal microscope after labelling with Oregon Green 488. The bar is 10 μm.

FIG. 2: myristic acid/CTACl vesicles r=0.65 c=1.01% diluted by three then dialysed for 5 days, observation with confocal microscope after labelling with Oregon Green 488. The bar is 10 μm.

FIG. 3: sodium myristate/N,N-dimethyltetradecylamine vesicles r=0.35 c=1.0% dialysed for 95 h, observation with confocal microscope after labelling with Rhodamine 6G.

FIG. 4: tetradecane dicarboxylic acid/cetyl trimethylammonium chloride vesicles r=0.49 c=1.0% dialysed for 4 days, observation with confocal microscope after labelling with Oregon Green.

FIG. 5: 16-hydroxy hexadecanoic acid/cetyl trimethylammonium chloride vesicles r=0.50 c=1.0% dialysed for 4 days, observation with confocal microscope after labelling with Oregon Green.

FIG. 6: myristic acid/CTACl vesicles r=0.66 c=1.00% diluted by three with encapsulated Rhodamine 6G dialysed for 5 days. The bar is 10 μm.

FIG. 7: myristic acid/CTACl vesicles r=0.65 c=1.01% diluted by three with encapsulated Rhodamine, dialysed for 5 days, observation with confocal microscope after labelling with Oregon Green 488.

FIG. 8: calibration curve of the response of Rhodamine B: F(576 nm)/F(600 nm) as a function of pH, imposed by adding HCl to a solution of Rhodamine B.

FIG. 9: pH in myristic acid/cetyltrimethyl ammonium chloride vesicles measured by the ratio of fluorescence of Rhodamine B at 546 nm and 600 nm, for different dilutions of the solution of vesicles.

FIG. 10: Variation of the concentration of Rhodamine 6G in the myristic acid/cetyl trimethylammonium chloride vesicles as a function of time, evaluated by the absorbance at 526 nm.

FIG. 11: Variation of the concentration of Oregon Green in myristic acid/cetyl trimethylammonium chloride vesicles as a function of time, evaluated by fluorescence at 522 nm with excitation at 502 nm.

FIG. 12: Variation of the mole fraction of myristic acid

$$r = \frac{c ( myristic )}{c ( myristic ) + c ( CTA )}$$

for mixtures with initial mole fraction r₀=0.32, 0.47, 0.55, 0.66 and 0.70 during the dialysis time.

FIG. 13: sodium myristate/N,N-dimethyltetradecylamine vesicles r=0.35 c=1.0% dialysed for 95 h, observation with confocal microscope one minute after adding Oregon Green.

FIG. 14: myristic acid/cetyl trimethylammonium chloride vesicles, r=0.66 c=1.0% diluted to 0.33% and dialysed for 4 days. Observation with the confocal microscope 20 minutes after adding Rhodamine 6G.

### EXAMPLE 1

### Preparation of Vesicles with Low Internal pH, Gel

Mix together 33.7 mg of myristic acid, 25.1 mg of cetyltrimethylammonium chloride and 5.7603 g of ultrapure water (r=0.65, c=1.01%). Stir at 50° C. for hours. The crystals of myristic acid gradually dissolve, giving a homogeneous white solution without solid residues.

Take about 3 mL of this solution and put it in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 1 h, 4 h, 24 h, 48 h and 5 days. Agitate the cassette vigorously before each change of dialysis water. Before the change of water at 1 h and 4 h of dialysis, demixing is observed, with a very viscous white solution as supernatant above a clear solution. After the change of water at 24 h of dialysis, demixing can no longer be observed, but the solution forms a very viscous gel.

After 5 days of dialysis, the membrane of the dialysis cassette is cut out, and the white gel is collected using a spatula and stored at room temperature.

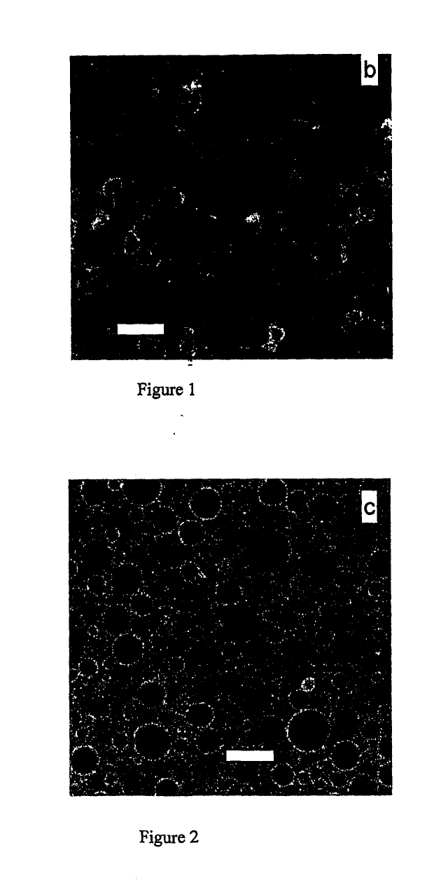

The presence of vesicles in the gel is checked by confocal microscopy after labelling with Oregon Green 488: about 100 mg of gel is taken, and 20 μL of a saturated aqueous solution of Oregon Green 488, diluted 10-fold, is added. Observation by confocal microscopy is carried out according to the usual techniques. An image of the vesicles is obtained, with the positive walls marked with the negative stain (FIG. 1). These vesicles form an almost compact stack.

### EXAMPLE 2

### Preparation of Vesicles with Low Internal pH, Fluid Solution

Mix together 33.7 mg of myristic acid, 25.1 mg of cetyltrimethylammonium chloride and 5.7603 g of ultrapure water (r=0.65, cᵢₙᵢₜᵢₐₗ=1.01%). Stir at 50° C. for 72 hours. The crystals of myristic acid gradually dissolve, giving a homogeneous white solution without solid residues.

Take 1.0122 g of this solution. Add 2.0513 g of water at 50° C. and stir for 30 seconds (c_{final}=0.33%). Put the mixture in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 1 h, 4 h, 24 h, 48 h and 5 days. Agitate the cassette vigorously before each change of dialysis water. During dialysis, the solution remains white, homogeneous and fluid.

After 5 days of dialysis, withdraw the solution from the cassette using a syringe and store it at room temperature.

The presence of vesicles in the solution is checked by confocal microscopy after labelling with Oregon Green 488: take about 100 mg of solution, and add 20 μL of a saturated aqueous solution of Oregon Green 488, diluted 10-fold. Observation by confocal microscopy is carried out according to the usual techniques. An image of the vesicles is obtained, with the positive walls marked with the negative stain (FIG. 2). The vesicles are well separated. The excess of negative stain remains in solution but does not penetrate into the vesicles.

### EXAMPLE 3

### Preparation of Vesicles with High Internal pH, Fluid Solution

Mix together 29.2 mg of sodium myristate, 53.4 mg of N,N-dimethyltetradecylamine and 8.1749 g of ultrapure water (r=0.35, c=1.00%). Stir at 50° C. for hours. The crystals of sodium myristate gradually dissolve and the oily droplets of N,N-dimethyltetradecylamine gradually disappear, giving a homogeneous white solution.

Take about 3 mL of this solution and put it in a 3-mL dialysis cassette with a cellulose membrane (Slide-a-lyzer, ThermoScientific) with cut-off threshold of 3500 Da. Immerse the cassette in a dialysis bath of about 500 mL with stirring at room temperature. Change the dialysis water for ultrapure water after 30 minutes, 1 h, 3 h, 7 h, 22 h, 95 h. Agitate the cassette vigorously before each change of dialysis water. Before the change of water at 30 minutes, 1 h, 3 h, 7 h and 22 h of dialysis, demixing is observed, into a white solution as supernatant above a clear solution. After the change of water at 22 h of dialysis, demixing can no longer be observed.

After 95 h of dialysis, the fluid white solution is taken from the dialysis cassette by means of a syringe and stored at room temperature.

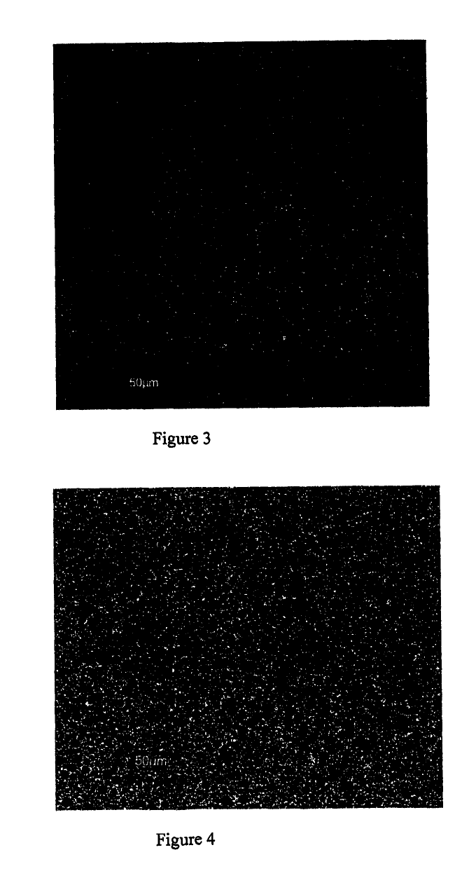

The presence of vesicles in the solution is checked by confocal microscopy after adding Rhodamine 6G: take about 100 μL of solution, and add 10 μL of an aqueous solution of Rhodamine 6G at 10 μM. Observation by confocal microscopy is carried out according to the usual techniques. An image of the impermeable vesicles is obtained, which appear in black against the continuous background of Rhodamine 6G in the external solution (FIG. 3).

### EXAMPLE 4

### Preparation of Vesicles with a Fatty Diacid

Mix together 20 mg of tetradecane dicarboxylic acid, 25.8 mg of cetyltrimethylammonium chloride and 4.5728 g of ultrapure water (r=0.49, c=1.00%). Stir at 50° C. for 75 hours. The crystals of dicarboxylic acid gradually dissolve, giving a homogeneous white solution without solid residues.

Take about 3 mL of this solution and put it in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 18 h, 27 h, 42 h, 66 h and 4 days. Agitate the cassette vigorously before each change of dialysis water. After the change of water at 27 h of dialysis, the solution forms a very viscous gel.

After 4 days of dialysis, the membrane of the dialysis cassette is cut out, and the white gel is collected using a spatula and stored at room temperature.

The presence of vesicles in the gel is checked by confocal microscopy after labelling with Oregon Green 488: take about 100 mg of gel, and add 20 μL of a saturated aqueous solution of Oregon Green 488, diluted 10-fold. Observation by confocal microscopy is carried out according to the usual techniques. An image of the vesicles is obtained, with the positive walls marked with the negative stain (FIG. 4). These vesicles form an almost compact stack.

### EXAMPLE 5

### Preparation of Vesicles with a Hydroxylated Fatty Acid

Mix together 22.0 mg of 16-hydroxy hexadecanoic acid, 26.3 mg of cetyltrimethylammonium chloride and 4.8263 g of ultrapure water (r=0.50, c=1.00%). Stir at 50° C. for 75 hours. The crystals of the dicarboxylic acid gradually dissolve, giving a homogeneous white solution without solid residues.

Take about 3 mL of this solution and put it in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 18 h, 27 h, 42 h, 66 h and 4 days. Agitate the cassette vigorously before each change of dialysis water. After the change of water at 27 h of dialysis, the solution forms a very viscous gel.

After 4 days of dialysis, the membrane of the dialysis cassette is cut out, and the white gel is collected using a spatula and stored at room temperature.

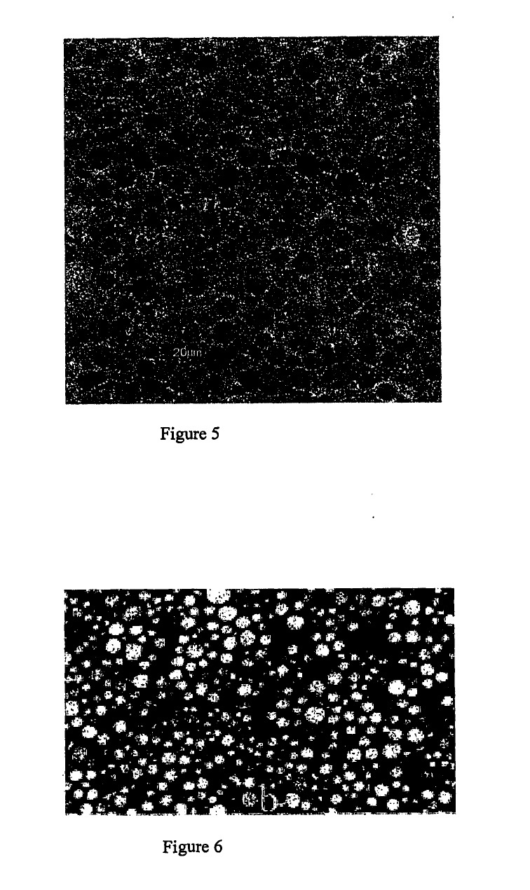

The presence of vesicles in the gel is checked by confocal microscopy after labelling with Oregon Green 488: take about 100 mg of gel, and add 20 μL of a saturated aqueous solution of Oregon Green 488, diluted 10-fold. Observation by confocal microscopy is carried out according to the usual techniques. An image of the vesicles is obtained, with the positive walls marked with the negative stain (FIG. 5). These vesicles form an almost compact stack.

### EXAMPLE 6

### Encapsulation of Organic Solutes in Vesicles

Mix together 33.8 mg of myristic acid, 25.1 mg of cetyltrimethylammonium chloride and 5.76 of solution of Rhodamine 6G at 4.5 mM in water (r=0.66, cᵢₙᵢₜᵢₐₗ=1.01%). Stir at 50° C. for 72 hours. The crystals of myristic acid gradually dissolve, giving a homogeneous solution without solid residues, which has the pink colour characteristic of Rhodamine 6G.

Take 1.67 g of this solution, make it up to 5.00 g by adding ultrapure water, and stir for 30 seconds (c_{final}=0.33%), [Rhodamine 6G]_{final}=1.5 mM). Put about 3 mL of this mixture in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 2 hours, 5 hours, 1 day, 2 days, days and 7 days. Agitate the cassette vigorously before each change of dialysis water. During dialysis, the solution remains homogeneous and fluid and has the pink coloration characteristic of Rhodamine 6G. Before the dialysis water is changed, the bath also has the pink coloration characteristic of Rhodamine 6G, but it gradually fades as the changes are made, until it becomes imperceptible at the change at 2 days of dialysis.

After 7 days of dialysis, withdraw the solution from the cassette using a syringe and store it at room temperature.

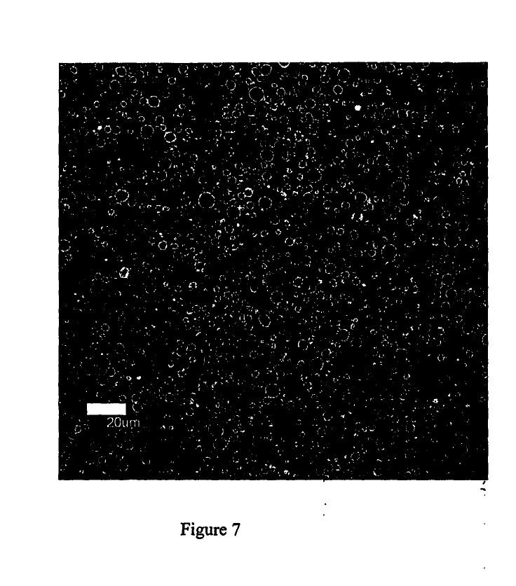

The presence of vesicles in the solution and encapsulation of Rhodamine 6G is checked by confocal microscopy according to the usual techniques. Rhodamine 6G is certainly present inside the vesicles, and exclusively inside (FIG. 7). The same observation can be carried out after labelling with Oregon Green 488: to 20 μL of solution, add 10 μL of a saturated aqueous solution of Oregon Green 488, diluted 10-fold. By using filters that are selective for Oregon Green and Rhodamine 6G, it is possible to localize each stain selectively: Oregon Green (FIG. 7) accumulates near the positively charged walls of the vesicles, Rhodamine 6G (FIGS. 6 and 7) is certainly present inside the vesicles, and exclusively inside.

### EXAMPLE 7

### Measurement of Internal pH for Vesicles with Low pH

Mix together 33.8 mg of myristic acid, 25.1 mg of cetyltrimethylammonium chloride and 5.76 g of solution of Rhodamine B at 10 μM in water (r=0.66, cᵢₙᵢₜᵢₐₗ=1.00%). Stir at 50° C. for 72 hours. The crystals of myristic acid gradually dissolve, giving a homogeneous white solution without solid residues.

Take 1.67 g of this solution, make up to 5.00 g by adding ultrapure water, and stir for 30 seconds (c_{final}=0.33%, [Rhodamine B]_{final}=3.33 μM). Put the mixture in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 2 hours, 5 hours, 1 day, 2 days, 4 days and days. Agitate the cassette vigorously before each change of dialysis water. During dialysis, the solution remains homogeneous and fluid and has the pink coloration characteristic of Rhodamine B. Before the dialysis water is changed, the bath also has the pink coloration characteristic of Rhodamine B, but it gradually fades as the changes are made, until it becomes imperceptible at the change at 1 day of dialysis.

After 7 days of dialysis, withdraw the solution from the cassette using a syringe and store it at room temperature. Observation of the solution by confocal microscopy according to the usual techniques without supplementary addition of other stains shows that the vesicles are well formed and that Rhodamine B is exclusively encapsulated inside the vesicles, not outside.

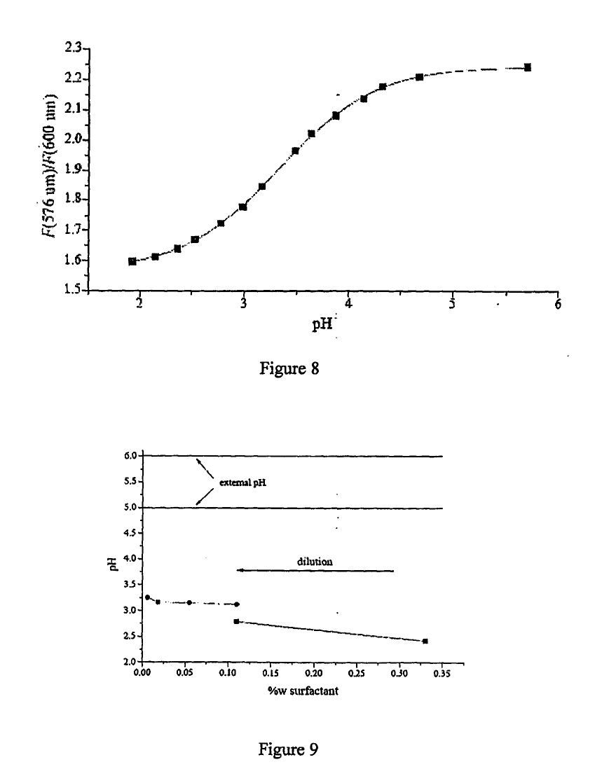

Using a fluorescence spectrometer (Varian Cary Eclipse), the solution is excited at 555 nm and measurements are taken to check that the ratio of fluorescence intensities at 576 nm and 600 nm is F(565 nm)/F(600 nm)=1.65. This value is compared with the calibration curve constructed by measuring the ratio of the fluorescence intensities measured in the same conditions in Rhodamine B/HCl mixtures of known pH (FIGS. 8 and 9). It is found that the internal pH of the vesicles is about pH=2.5, whereas the pH of the solution measured by means of a pH electrode immersed directly in the solution of vesicles gives a pH equal to 6. Dilution of the solution up to a factor of 80 does not destroy the vesicles, and makes it possible to keep a pH inside the vesicles below 3.5. The excess concentration of protons within the vesicles is maintained over periods of several months even when the vesicles are left under dialysis (Table 1).

TABLE 1
Intravesicular pH and associated concentration as a
function of dialysis time, measured by the ratio of
fluorescence of Rhodamine B at 546 nm and 600 nm.

| Dialysis time | | Intravesicular pH | Corresponding c(H⁺) |
|---|---|---|---|
| 7 | days | 3.15 | 0.71 mM |
| 18 | days | 3.31 | 0.48 mM |
| 2 | months | 3.48 | 0.34 mM |

### EXAMPLE 8

### Measurement of Permeability to Cations and of the Degree of Encapsulation

Mix together 33.8 mg of myristic acid, 25.1 mg of cetyltrimethylammonium chloride and 5.76 of solution of Rhodamine 6G at 4.5 mM in water (r=0.66, cᵢₙᵢₜᵢₐₗ=1.01%). Stir at 50° C. for 72 hours. The crystals of myristic acid gradually dissolve, giving a homogeneous solution without solid residues, which has the pink colour characteristic of Rhodamine 6G.

Take 1.67 g of this solution, make up to 5.00 g by adding ultrapure water and stir for 30 seconds (c_{final}=0.33%, [Rhodamine 6G]_{final}=1.5 mM). Put about 3 mL of this mixture in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 2 hours, 5 hours, 1 day, 2 days, days and 7 days. Agitate the cassette vigorously before each change of dialysis water. During dialysis, the solution remains homogeneous and fluid and has the pink coloration characteristic of Rhodamine 6G. Before the dialysis water is changed, the bath also has the pink coloration characteristic of Rhodamine 6G, but it gradually fades as the changes are made, until it becomes imperceptible at the change at 2 days of dialysis.

Without interrupting dialysis, take about 200 μL of solution after 7 days, 14 days, 21 days, 28 days, 45 days and 57 days of dialysis. After taking the last sample, measure the residual amount of Rhodamine 6G in each sample as follows: the vesicles are destroyed by 40 μL of a solution of Triton X-100 at 5 wt. % in water, added to 200 μL of sample and heated at 50° C. for a few minutes. The complete destruction of the vesicles is verified by confocal microscopy. The solution is made up to 3.5 mL by adding ultrapure water, and the absorbance of the solution at 526 nm is measured using a UV-visible spectrometer (Varian Cary 1). The concentration of Rhodamine 6G in each sample is proportional to the absorbance. Therefore the concentration of Rhodamine 6G in the vesicles is plotted as a function of the dialysis time and the adjustment of the curve by an exponential law gives the permeability according to the equation:

$${c}_{solute} ( t ) = {c}_{0 , solute} {e}^{- \frac{3 {P}_{solute}}{R} t}$$

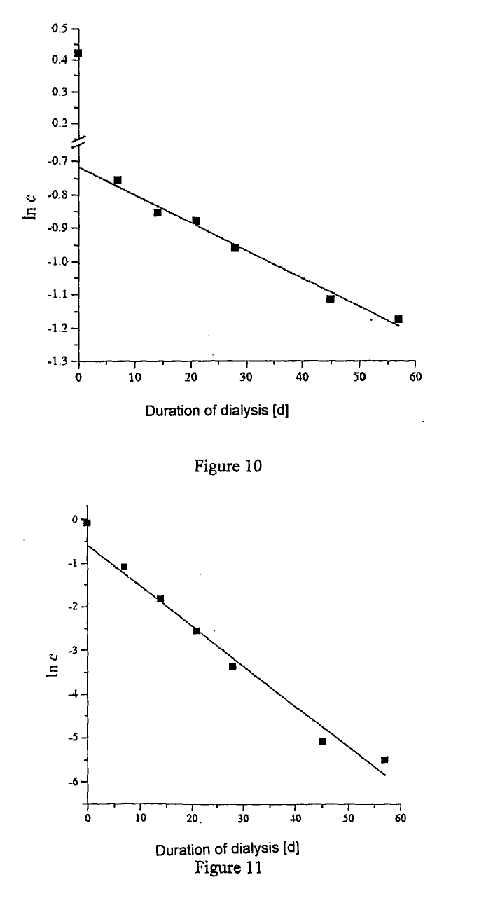

(FIG. 10). The radius R of the vesicles is estimated by confocal microscopy at 2.5 μm, which finally allows us to deduce that the permeability of the vesicles to Rhodamine 6G is 8.1 10⁻¹² cm/s. The ratio of the initial concentration of Rhodamine 6G in the solution (1.5 mM) to the concentration in the vesicles extrapolated to t=0 (0.50 mM) provides a measure of the degree of encapsulation. 30% of the initial Rhodamine 6G is therefore encapsulated.

### EXAMPLE 9

### Measurement of the Permeability to Anions and of the Degree of Encapsulation

Mix together 33.8 mg of myristic acid, 25.1 mg of cetyltrimethylammonium chloride and 5.76 of saturated solution of Oregon Green in water (r=0.66, cᵢₙᵢₜᵢₐₗ=1.01%). Stir at 50° C. for 72 hours. The crystals of myristic acid gradually dissolve, giving a homogeneous solution without solid residues which has the characteristic greenish-yellow colour of Oregon Green.

Take 1.67 g of this solution, make up to 5.00 g by adding ultrapure water and stir for 30 seconds (c_{final}=0.33%). Put about 3 mL of this mixture in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 2 hours, 5 hours, 1 day, 2 days, 4 days and 7 days. Agitate the cassette vigorously before each change of dialysis water. During dialysis, the solution remains homogeneous and fluid and has the characteristic yellow-green coloration of Oregon Green. Before the dialysis water is changed, the bath also has the characteristic yellow-green coloration of Oregon Green, but it gradually fades as the changes are made, until it becomes imperceptible at the change at 1 day of dialysis.

Without interrupting dialysis, take about 200 μL of the solution after 7 days, 14 days, 21 days, 28 days, 45 days and 57 days of dialysis. After taking the last sample, the residual amount of Oregon Green in each sample is measured as follows: the vesicles are destroyed by adding 40 μL of a solution of Triton X-100 at 5 wt. % in water to 200 μL of sample and heating at 50° C. for a few minutes. Complete destruction of the vesicles is verified by confocal microscopy. The solution is made up to 3 mL by adding a solution of CTACl at 1.5 mM in a borate buffer solution at pH=9. The fluorescence emission at 522 nm with excitation at 502 nm is measured with a Varian Cary Eclipse fluorescence spectrometer for all the solutions prepared from each of the samples taken. The concentration of Oregon Green in each sample is proportional to the fluorescence emission to within an arbitrary factor that is not important for the measurement. Therefore the fluorescence is plotted as a function of the dialysis time and adjustment of the curve by an exponential law gives the permeability according to the equation:

$$F ( t ) = {F}_{0} {e}^{- \frac{3 {P}_{solute}}{R} t}$$

(FIG. 11). The radius R of the vesicles is estimated by confocal microscopy at 2.5 μm, finally allowing us to deduce that the permeability of the vesicles to Oregon Green is 8.9 10⁻¹¹ cm/s. The ratio of the fluorescence measured from the initial solution to the fluorescence extrapolated to t=0 makes it possible to measure the degree of encapsulation. 60% of the initial Oregon Green is therefore encapsulated.

### EXAMPLE 10

### HPLC Measurement of the Mole Fraction of Surfactant During Dialysis

The myristic acid/cetyl trimethylammonium chloride mixture to be analysed is diluted in 4 times its mass of absolute ethanol. The concentrations of myristic acid and of cetyl trimethylammonium in this solution are measured by HPLC using a C18 HyPurity column coupled to a Sedex 75 refractometer conditioned at 40° C. under a nitrogen pressure of 2.9 bar. The continuous phase for the measurement is a 70/15/15 mixture by volume of acetone/methanol/water acidified with 0.06 vol. % of trifluoroacetic acid. The mixture is injected at 0.5 mL/min. In these conditions, cetyl trimethylammonium and myristic acid produce peaks that are well separated after migration times of 3.5 and 6 minutes respectively. The area under these peaks is compared with calibration curves constructed using solutions of cetyl trimethylammonium chloride and myristic acid prepared in the same water/ethanol ratio, and of known concentrations.

This technique makes it possible to trace the evolution of the mole fraction

$$r = \frac{c ( myristic )}{c ( myristic ) + c ( CTA )}$$

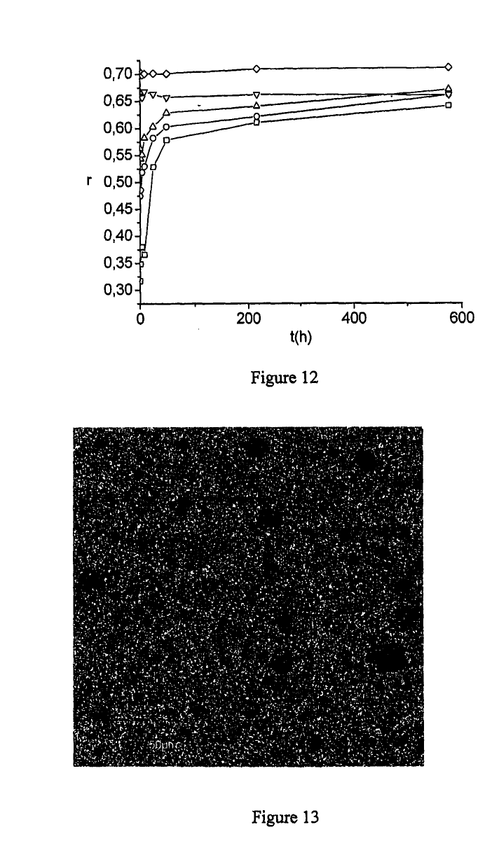

of myristic acid in any mixture of myristic acid and cetyl trimethylammonium chloride of any initial mole fraction and any dialysis time (FIG. 12).

### EXAMPLE 11

### Spontaneous Encapsulation of a Weak Acid in a Vesicle at High pH

Mix together 29.2 mg of sodium myristate, 53.4 mg of N,N-dimethyltetradecylamine and 8.1749 g of ultrapure water (r=0.35, c=1.00%). Stir at 50° C. for hours. The crystals of sodium myristate gradually dissolve and the oily droplets of N,N-dimethyltetradecylamine gradually disappear, giving a homogeneous white solution.

Take about 3 mL of this solution and put it in a 3-mL dialysis cassette with a cellulose membrane (Slide-a-lyzer, ThermoScientific) with cut-off threshold of 3500 Da. Immerse the cassette in a dialysis bath of about 500 mL with stirring at room temperature. Change the dialysis water for ultrapure water after 30 minutes, 1 h, 3 h, 7 h, 22 h, 95 h. Agitate the cassette vigorously before each change of dialysis water. Before the change of water at 30 minutes, 1 h, 3 h, 7 h and 22 h of dialysis, demixing is observed between a white solution as supernatant above a clear solution. After the change of water at 22 h of dialysis, demixing can no longer be observed.

After 95 h of dialysis, the fluid white solution is withdrawn from the dialysis cassette by means of a syringe and stored at room temperature.

The presence of vesicles in the solution is checked by confocal microscopy by adding Rhodamine 6G as described above. The spontaneous encapsulation of Oregon Green is effected as follows: add 10 μL of saturated solution of Oregon Green in water, diluted 10-fold, to 100 μL of the solution of vesicles. After a waiting time of one minute, confocal microscopy is able to detect zones with high concentrations of Oregon Green (FIG. 13) which corresponds to the spontaneous encapsulation of the slightly acid stain in the high-pH interior of the vesicles.

### EXAMPLE 12

### Spontaneous Encapsulation of Cationic Solutes in Vesicles

Mix together 33.8 mg of myristic acid, 25.1 mg of cetyltrimethylammonium chloride and 5.76 g of ultrapure water (r=0.66, cᵢₙᵢₜᵢₐₗ=1.01%). Stir at 50° C. for 72 hours. The crystals of myristic acid gradually dissolve, giving a homogeneous white solution without solid residues.

Take 1.67 g of this solution, make up to 5.00 g by adding ultrapure water and stir for 30 seconds (c_{final}=0.33). Put about 3 mL of this mixture in a 3-mL dialysis cassette with a cellulose membrane (Slide-a-lyzer, ThermoScientific) with cut-off threshold of 3500 Da. Immerse the cassette in a dialysis bath of about 300 mL with stirring at room temperature. Change the dialysis water for ultrapure water after 2 hours, 5 hours, 1 day, 2 days and 4 days. Agitate the cassette vigorously before each change of dialysis water. During dialysis, the solution remains homogeneous and fluid.

After 4 days of dialysis, withdraw the solution from the cassette using a syringe and store it at room temperature.

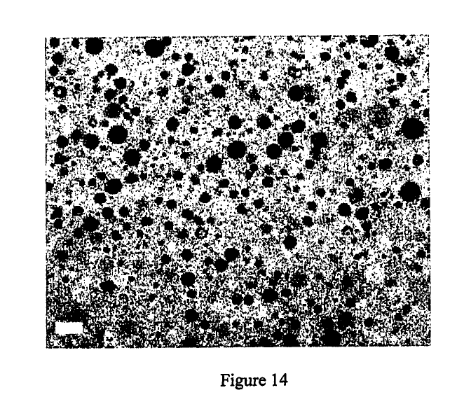

The spontaneous encapsulation of Rhodamine 6G is effected as follows: add about 10 μL of Rhodamine 6G at 10 μM to 20 μL of the solution of vesicles. After 20 minutes, zones with high concentration of Rhodamine 6G are observed by confocal microscopy (FIG. 14). They correspond to the interior of the vesicles in which Rhodamine 6G has accumulated preferentially.

### EXAMPLE 13

### Formation of Vesicles by Dialysis at Low pH

Mix together 33.8 mg of myristic acid, 25.1 mg of cetyltrimethylammonium chloride and 5.76 g of ultrapure water (r=0.66, cᵢₙᵢₜᵢₐₗ=1.01%). Stir at 50° C. for 72 hours. The crystals of myristic acid gradually dissolve, giving a homogeneous white solution without solid residues.

Put about 3 mL of this mixture in a 3-mL dialysis cassette with a cellulose membrane (Slide-a-Lyzer, ThermoScientific) with cut-off threshold of 3500 Da. Immerse the cassette in a dialysis bath of about 300 mL of HCl at 10⁻⁴ M with stirring at room temperature. Replace the dialysis solution with a solution of HCl at 10⁻⁴ M after 2 hours, 5 hours, 1 day, 2 days and 4 days. Agitate the cassette vigorously before each change of dialysis solution. During dialysis, the solution of surfactants eventually forms a homogeneous white gel.

After 4 days of dialysis, the gel is taken from the cassette and stored at room temperature. The gel is formed of vesicles of myristic acid and cetyltrimethylammonium.

### EXAMPLE 14

### Formation of Vesicles by Dialysis at High pH

Mix together 33.8 mg of myristic acid, 25.1 mg of cetyltrimethylammonium chloride and 5.76 g of ultrapure water (r=0.66, cᵢₙᵢₜᵢₐₗ=1.01%). Stir at 50° C. for 72 hours. The crystals of myristic acid gradually dissolve, giving a homogeneous white solution without solid residues.

Put about 3 mL of this mixture in a 3-mL dialysis cassette with a cellulose membrane (Slide-a-Lyzer, Thermo Scientific) with cut-off threshold of 3500 Da. Immerse the cassette in a dialysis bath of about 300 mL of NaOH at 10⁻⁴ M with stirring at room temperature. Replace the dialysis solution with a solution of NaOH at 10⁻⁴ M after 2 hours, 5 hours, 1 day, 2 days and 4 days. Agitate the cassette vigorously before each change of dialysis solution. During dialysis, the solution of surfactants eventually forms a homogeneous white gel.

After 4 days of dialysis, the gel is taken from the cassette and stored at room temperature. The gel is formed of vesicles of myristic acid and cetyltrimethylammonium.

1. A non-porous vesicle, dispersed in an aqueous composition which defines an external phase, wherein a wall of the vesicle, which defines an internal phase, comprises a mixture comprising:
at least one anionic surfactant with a counter-ion A⁺; and
at least one cationic surfactant with a counter-ion B⁻,
which are co-crystallized and are an ion pair,
wherein the ion pair, A⁺B⁻, is different from the ion pair, H⁺OH⁻, and with at least A⁺=H⁺ or B⁻═OH⁻.

2. The vesicle of claim 1, having a permeability to H⁺ or to OH⁻ which is less than 5.10⁻¹¹ cm/s.

3. The vesicle of claim 1, having a pH difference of at least 2 units between the internal phase and the external phase,
wherein the difference is maintained for a duration of at least 1 month.

4. The vesicle of claim 1, wherein the cationic surfactant is at least one cationic surfactant comprising a cationic polar group and at least one saturated or unsaturated alkyl or aryl or aralkyl chain, having from 8 to 30 carbon atoms and optionally interrupted by an ester or ether function, and
wherein the anionic surfactant is at least one anionic surfactant comprising an anionic polar group and at least one C₈-C₃₀ alkyl chain optionally substituted with one or more hydroxyl groups and one or more halogen atoms.

5. The vesicle of claim 1, wherein the cationic surfactant is at least one selected from the group consisting of a monocatenary ammonium of formula (I) and a bicatenary ammonium of formula (I′), and the corresponding bases: 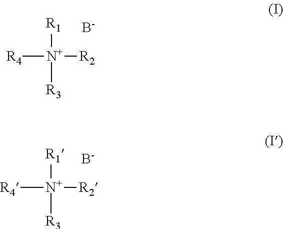
wherein:
R₁, R₂, R₃, R′₁, and R′₂, which are identical or different, represent H, a C₁-C₄ alkyl, a C₁-C₄ hydroxyalkyl, or an alkyl(C₁-C₄)ether,
R₄, R′₃, and R′₄, which are identical or different, represent a saturated or unsaturated C₈-C₂₄ hydrocarbon chain, a benzyl, a C₈-C₂₄ aralkyl ester of alkyl(C₄-C₂₀) group, or an alkyl(C₄-C₂₀)ester of alkyl(C₄-C₂₀) group,
B⁻ represents OH⁻, F⁻, Cl⁻, Br⁻, I⁻, BF₄⁻, or SO₄²⁻,
and the anionic surfactant is at least one selected from a carboxylic acid with a C₈-C₂₄ alkyl a carboxylic acid with a C₈-C₁₄ alkyl hydroxyalkyl chain, a carboxylic acid with a C₈-C₂₄ fluoroalkyl chain, a dicarboxylic acid with a hydrophobic C₈-C₂₄ carbon chain, a phosphate having one or two C₈-C₂₄ alkyl groups, a sulphate having one or two C₈-C₂₄ alkyl groups, and a sulfonate having one or two C₈-C₂₄ alkyl chains.

6. The vesicle of claim 1, with an acid internal phase, wherein the anionic surfactant has the H⁺ counter-ion and the cationic surfactant has the B⁻ counter-ion,
wherein B⁻ being is different from OH⁻.

7. The vesicle of claim 1, with a basic internal phase, wherein the anionic surfactant has the A⁺ counter-ion, A⁺ being different from H⁺ and the cationic surfactant has the OH⁻ counter-ion.

8. The vesicle of claim 6, wherein:
the anionic surfactant is at least one acid or salt thereof selected from the group consisting of: 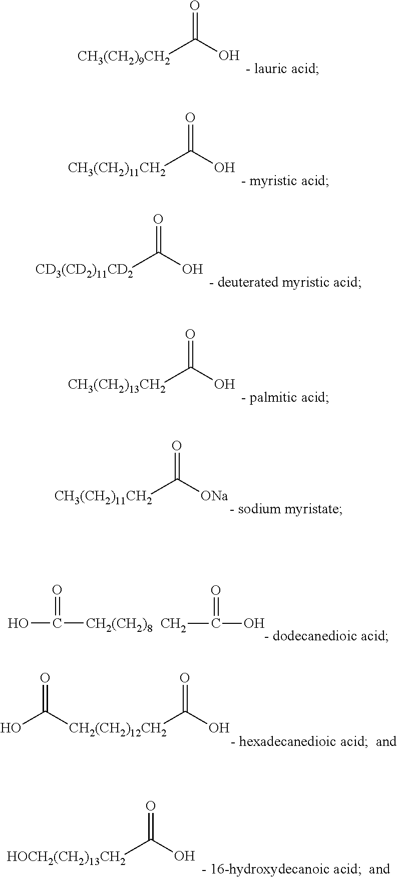
wherein the cationic surfactant is at least one selected from the group consisting of:

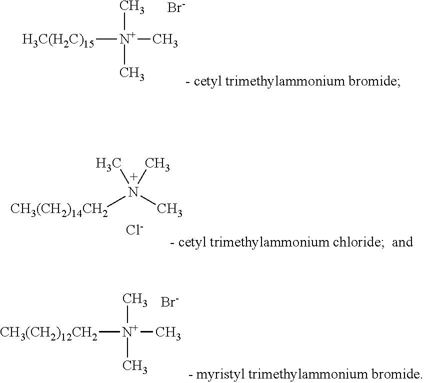

9. The vesicle of claim 7, wherein the anionic surfactant is at least one selected from the group consisting of:
sodium myristate, sodium tetradecane sulphonate, and sodium hexadecane sulphonate; and
wherein the cationic surfactant is at least one amine of salt thereof selected from the group consisting of the:
tridecylamine, tetradecylamine, hexadecylamine, N,N-dimethyltridecylamine, and N,N-dimethyltetradecylamine.

10. The vesicle of claim 1, having a size of 0.1-100 μm.

11. The vesicle of claim 1, having an active principle encapsulated in their internal cavity.

12. A method for preparing a dispersion of at least one vesicle of claim 1 in an aqueous medium, the method comprising:
i) mixing, in water, at least one anionic surfactant with a counter-ion A⁺ and at least one cationic surfactant with a counter-ion B⁻, wherein the ion pair, A⁺, B⁻ is different from the ion pair, H⁺, OH⁻, and with at least A⁺=H⁺ or B⁻═OH⁻, to obtain a mixture;
ii) heating the mixture, for a time, tₕₑₐₜ, at a temperature, T, below a melting point of the mixture and which is greater than or equal to 40° C. but below 90° C., wherein T and tₕₑₐₜ is sufficient to obtain dissolution of the surfactants, to obtain a suspension;
iii) subjecting the suspension obtained in ii) to a dialysis so as to remove at least one salt from the external phase.

13. The method of claim 12, wherein the aqueous medium is water.

14. The method of claim 12, wherein a mole fraction of anionic surfactant is calculated from the formula:
$${r}_{A} = \frac{{n}_{A}}{{n}_{A} + {n}_{C}}$$
wherein n_{A} and n_{C} denotes respectively the number of moles of anionic surfactant and the number of moles of cationic surfactant,
the fraction r_{A} being selected for each pair of surfactants, or mixtures of surfactants according to a protocol comprising:
a) mixing the anionic and cationic surfactants in water, selecting a fraction r_{A1} at random, to obtain a mixture;
b) heating the mixture at a temperature below the melting point of the anionic surfactant, for a time greater than or equal to one hour, to obtain a suspension;
c) submitting the suspension obtained to dialysis;
d) during dialysis, measuring a variation of r_{A1} in the suspension, optionally by monitoring the concentration of the surfactants by HPLC;
e) observing that r_{A1} reaches a limit value that no longer changes and is independent of the value of r_{A1} chosen initially;
f) calling this value r_{1(A)}; and
g) preparing the vesicles a with value of r_{A} greater than r_{1(A)}.

15. The method of claim 12, wherein a mole fraction of cationic surfactant is calculated from the formula:
$${r}_{C} = \frac{{n}_{C}}{{n}_{A} + {n}_{C}}$$
wherein the fraction r_{C} is selected for each pair of surfactants, or mixtures of surfactants according to a protocol comprising:
a) mixing together the anionic and cationic surfactants in water, selecting a fraction r_{C1} at random, to obtain a mixture;
b) heating the mixture at a temperature below the melting point of the cationic surfactant T_{C}, for a time greater than or equal to one hour, to obtain a suspension;
c) submitting the suspension obtained to dialysis;
d) during dialysis, measuring the variation of r_{C1} in the suspension, optionally by monitoring the concentration of the surfactants by HPLC;
e) observing that r_{C1} reaches a limit value which no longer changes and which is independent of the value of r_{C1} chosen initially;
f) calling this value r_{1(C)}; and
g) preparing the vesicles with a value of r_{C} greater than r_{1(C)}.

16. The method of claim 12, wherein a concentration of surfactants in the mixture is between 0.01 and 30%.

17. The method of claim 12, wherein T is greater than or equal to 40° C. and less than or equal to 80° C.

18. The method of claim 12, wherein tₕₑₐₜ is between one hour and one week.

19. The method of claim 12, wherein a solution comprising an active substance to be encapsulated is added to the suspension following the heating ii).

20. The method of claim 12, wherein an active substance to be encapsulated is added to the suspension following the dialysis iii).

21. The method of claim 12, wherein the vesicles are stabilized by at least one polymer adsorbed on their surface.

22. An active principle, encapsulated by the vesicle of claim 1, wherein the active principle is a water-soluble organic molecule.

23. A method of decontaminating an aqueous environment, the method comprising contacting the aqueous environment with the vesicle of claim 1.

24. A cosmetic composition, comprising at least one vesicle of claim 1.

25. A pharmaceutical composition, comprising at least one vesicle of claim 1.
